(19) United States
(12) Patent Application Publication
(10) Pub. No.: US 2007/0293441 A1
(43) Pub. Date: Dec. 20, 2007

(54) HIGH-PRESSURE STERILIZATION TO TERMINALLY STERILIZE PHARMACEUTICAL PREPARATIONS AND MEDICAL PRODUCTS

(75) Inventors: Carolyn Choo, Long Grove, IL (US); Dirk Faict, Assenede (BE); Alfredo Rodriquez, Arlington Heights, IL (US)

Correspondence Address: BAXTER HEALTHCARE CORPORATION, 1 BAXTER PARKWAY, DF2-2E, DEERFIELD, IL, 60015, US

(73) Assignees: Baxter International Inc., Deerfield, IL (US); Baxter Healthcare S.A., Wallisellen (CN)

(21) Appl. No.: 11/766,471

(22) Filed: Jun. 21, 2007

Related U.S. Application Data
(63) Continuation-in-part of application No. 10/946,885, filed on Sep. 22, 2004.
(60) Provisional application No. 60/505,235, filed on Sep. 22, 2003.

(51) Int. Cl. A61K 31/70; A61L 2/00; A61P 43/00

(52) U.S. Cl. 514/23; 422/33

(57) ABSTRACT

The present disclosure provides a sterilized medical system that includes an ultra high pressure sterilized glucose solution in a container. The sterile glucose solution may contain less than about 40 ppm total glucose degradation product. The glucose solution may be a ready-to-use infusion solution disposed in a single chamber container.

This application is a continuation-in-part of U.S. patent application Ser. No. 10/946,885 filed on Sep. 22, 2004, which claims priority from U.S. Provisional Application No. 60/505,235 filed on Sep. 22, 2003.

### BACKGROUND

The present disclosure provides a process for sterilizing medical systems, such as medical solutions, in suitable containers using high pressure sterilization techniques.

Medical systems, and particularly medical solutions, often require sterilization prior to use. As used herein, “medical solutions” includes, without limitation, solutions, suspensions, and emulsions containing pharmaceutical ingredients; solutions for renal application (such as dialysis fluids) and other forms of pharmaceutical preparations such as carbohydrate solutions, amino acid solutions and lipid emulsions (and mixtures thereof) for parenteral nutrition. Other examples of medical system components include medical device disposables, such as pharmaceutical-containing bags (often made of plasticized PVC or other plastics), blood bags, dialyzers, systems for use on automated devices (e.g., blood separation devices, infusion pumps, etc.). Such systems may be sensitive to traditional sterilization techniques such as gamma sterilization, ethylene oxide sterilization or autoclaving. For example, some glucose-containing medical solutions are subject to glucose degradation or aggregation when sterilized using conventional moist heat sterilization techniques. Similarly, a number of medical solutions contain peptides or proteins that are susceptible to degradation if they are exposed to high temperatures for a prolonged duration, as is the case during conventional moist heat sterilization. It is also the case that certain medical devices, such as medical solution containers, may contain components or structures that cannot withstand prolonged exposure to high temperatures during sterilization. For example, the peelable seals in certain multiple-chamber containers may strengthen or even become permanent when exposed to high temperature. Similarly, certain barrier films exhibit a significant reduction in barrier properties during and after high temperature sterilization, allowing moisture and/or oxygen to permeate the container. A need therefore exists for improved techniques for sterilizing a medical system without compromising its integrity or suitability for the intended therapeutic use.

### SUMMARY

The present disclosure provides a method for sterilizing medical systems. Such systems may include, without limitation, compositions, medical solutions, containers for medical solutions, medical devices, and combinations of the above. The method facilitates effective sterilization without significantly diminishing the efficacy of such systems. The disclosure further provides sterilized medical solutions. Suitable containers include any container that is stable under the present method including medical delivery devices containing medical solutions.

The method involves heating the system and pressurizing the system in excess of 0.25 MPa for a period of time sufficient to make the system sterile. In an embodiment, the system will achieve a temperature in excess of 70° C. The steps of supplying heat and pressure are preferably carried out simultaneously for at least a sufficient period of time to sterilize the system. The system can then be allowed to return to ambient temperature and pressure for storage, shipment or use.

The method can be used on empty containers or containers containing any of a wide variety of medical solutions. A medical solution may be considered any solution administered to a patient to achieve a therapeutic effect (i.e., alleviate or treat an ailment or disease) and includes, without limitation, pharmaceutical solutions, nutritional solutions, and dialysis solutions. Dialysis solutions include, without limitation, solutions for acute or chronic hemodialysis, hemofiltration or hemodiafiltration solutions for acute or chronic peritoneal dialysis, and solutions for ambulatory peritoneal dialysis, automated peritoneal dialysis and continuous renal replacement therapy. Such solutions may be administered to the patient by any known technique. Nonlimiting examples of suitable techniques may include parenteral administration routes such as intravenous, intramuscular or subcutaneous injection or infusion, intraperitoneal infusion, and other methods appropriate for the particular medical solution.

In an embodiment, a method of producing a sterile dialysis solution is provided. The method includes the steps of providing an aqueous dialysis solution containing an osmotic agent and a buffer in a flexible container; pressurizing the solution and container to a hydrostatic pressure of about 100 M[Pa to about 1500 MPa; and releasing the hydrostatic pressure, thereby producing a sterile solution within the container. In this embodiment, substantially all of the dialysis solution may be disposed within one compartment of the container during the pressurization step. Alternatively, the container may include separate compartments containing the osmotic agent and the buffer, the container being adapted to permit selective fluid communication between the compartments. For example, the container may include an internal conduit with a frangible seal between the compartments, or may include one or more peelable seals separating the compartments. In an embodiment, the dialysis solution is substantially free of oxidation inhibiting agents.

In an embodiment, another method of preparing a dialysis solution is provided. The method may include the steps of (1) providing first and second component solutions in discrete chambers of a multiple chamber container adapted to permit selective fluid communication between said chambers and (2) subjecting the multiple chamber container to a hydrostatic pressure from about 100 MPa to about 1500 MPa for I to about 300 seconds. The first component solution may contain up to about 50 weight percent glucose, glucose polymer, or a mixture thereof; in an embodiment, the first component solution may have a pH of about 1.5 to about 5.5. The second component solution may include a buffer concentrate; in an embodiment, the buffer concentrate may have a pH of about 6.0 to about 10.0. When mixed, the first and second component solutions form a ready-to-use dialysis solution having a pH of about 4.5 to about 8.0. The method may also include the step of preheating the container to a temperature of about 70° C. to about 99° C. before subjecting the container to the hydrostatic pressure.

In an embodiment, another method of preparing a dialysis solution is provided. The method may include the steps of providing first, second and third component solutions in discrete chambers of a multiple chamber container adapted to permit selective fluid communication between the chambers, and subjecting the multiple chamber container to a hydrostatic pressure from about 100 MPa to about 1500 MPa for 1 to about 300 seconds. In this embodiment, the first component solution may have a pH of about 3.0 to about 6.0 and contain up to about 50 weight percent glucose, glucose polymer, or a mixture thereof; the second component solution may include a buffer concentrate having a pH of about 6.5 to about 10.0; and the third component solution may have a pH of about 1.5 to about 4.5. When mixed, the first, second and third component solutions form a ready-to-use dialysis solution having a pH of about 4.5 to about 8.0. The method may also include the step of preheating the container to a temperature of about 70° C. to about 99° C. before subjecting the container to the hydrostatic pressure.

The methods disclosed herein are particularly useful in the sterilization of solutions that contain glucose. It is known that carbohydrates such as glucose can degrade during conventional heat sterilization procedures such as autoclaving to form toxic or otherwise undesirable glucose degradation products within the sterilized solution. By applying heat for a significantly reduced duration, ultra high pressure sterilization minimizes glucose degradation that occurs when glucose-containing medical solutions are exposed to high temperatures for the extended duration (e.g. 30-60 minutes) normally required to achieve sterilization. Thus, the method can be used to sterilize solutions containing glucose such that the glucose remains substantially undegraded. The glucose may be greater than about 75% undegraded after sterilization, for example greater than about 80% undegraded, greater than about 85% undegraded, greater than about 90% undegraded, or even greater than about 95% undegraded.

The disclosed methods are also useful in the sterilization of solutions that contain both sugars and amino acids. The components in these solutions are known to react with each other during the prolonged exposure to high temperatures that is associated with conventional moist heat sterilization methods.

In an embodiment, the methods may be used to prepare sterilized solutions in which the glucose component is substantially undegraded. For example, the medical solution may be a UMP sterilized dialysis solution. The dialysis solution may include glucose as an osmotic agent. The osmotic agent may be present in an amount from about 1% to about 50% by weight of the solution. The solution may have a pH from about 6.0 to about 8.0, for example from about 6.7 to about 7.5. The sterilized solution may contain less than about 45 ppm total glucose degradation product.

In an embodiment, the dialysis solution may be ready for administration—i.e., requiring no mixing of components, no addition of other components, and no further sterilization, prior to administration to a patient.

In an embodiment, the sterilized dialysis solution may be substantially precipitate-free. The sterilized dialysis solution may be clear and have no cloudiness and/or no turbidity.

In another embodiment, the medical system may include a terminally sterilized medical solution, for example a dialysis solution, in a single chamber container.

In a further embodiment, the present disclosure describes a method of treating renal disease. The method includes the step of providing a sterile dialysis solution in a flexible container, the solution and container having been pressurized together at a pressure of about 100 MPa to about 1500 MPa. The solution may include at least one osmotic agent, at least one buffer and at least one electrolyte. The method may further include the step of infusing the sterile solution into a patient having a need for such treatment. In certain embodiments, the flexible container contains a mixture of the buffer and osmotic agent during pressurization. For example, the container may have only one solution compartment containing the mixture. Alternatively, the container may include separate compartments for the osmotic agent and the buffer. In some embodiments, the solution is substantially free of oxidation inhibiting agents.

Accordingly, the methods and products contemplated by the present disclosure provide one or more of the following advantages:
a sterilization method that avoids the component degradation problems and container deformation problems associated with conventional sterilization processes and autoclave sterilization in particular;.
increased product quality in the form of minimized chemical degradation in medical solutions and glucose-containing solutions in particular;
reduced sterilization times for medical systems, even large-volume solutions;
sterilized glucose containing solutions with no or a very low levels of glucose degradation product;
terminally sterilized, ready-to-use glucose-containing dialysis solutions in a single chamber container;
infusion solutions that require no mixing or pre-mixing of segregated solution components;
dialysis solutions that do not require the glucose component to be segregated from other dialysis solution components prior to use.

These and other aspects and attributes of the present disclosure will be discussed with reference to the following drawings and accompanying specification.

### BRIEF DESCRIPTION OF THE FIGURES

FIG. 1 shows a pressure-time-temperature profile;

FIG. 2 shows a plan view of a flowable materials container;

FIG. 3 shows a plan view of a multiple chamber peel seal container and fluid administration set;

FIG. 4 shows a syringe;

FIG. 5 shows a cartridge for a medical delivery device;

FIG. 6 shows a fluid access device;

### DETAILED DESCRIPTION

While this disclosure sets forth embodiments of the invention in many different forms, there are shown in the drawings, and will be described herein in detail, embodiments thereof with the understanding that the present disclosure is to be considered as an exemplification of the principles set forth herein and is not intended to be limited to the specific embodiments illustrated.

The present disclosure provides a method for sterilizing a medical system or medical solution without significantly diminishing the usability, stability, and/or efficacy of the system/solution. The present disclosure provides a method of sterilizing a dynamic system (i.e., a system capable of going from a stable to an unstable state) wherein the system is subjected to high pressure for a time sufficient to sterilize the system without causing the system to go from a stable state to an unstable state.

As used herein, the term “sterilization” and its variants shall mean the kill or control of bacteria, virus, protozoa or other biological microbes in a system such that the system provides a reduced risk of infection upon use with a mammal, for example a human. Methods of the present disclosure shall sterilize a system to the point that all or nearly all of the biological microbes are killed or rendered non-replicating.

In an embodiment, the method may be used to sterilize a medical system. The medical preparation may be prepared by any of numerous techniques known in the art or that will be developed hereafter. In general, the method provides subjecting a medical system to ultra high pressure for a period of time sufficient to achieve sterilization. The method may be applied to medical solutions having heat sensitive components and/or small particle dispersions. The disclosure further provides for sterilized medical solutions.

The high-pressure sterilization techniques of the present disclosure allow for sterilization of medical solutions without causing significant degradation of the ingredients therein. Moreover, heat may be transferred instantaneously throughout the medical solution due to rapid adiabatic heating of the formulation during the pressurization step. It is anticipated that the high-pressure sterilization techniques are suitable for use with many medical solutions containing various ingredients such as pharmaceutical compounds in a number of container configurations.

In general, the method provides sterilizing a medical solution at ultra high pressure. The medical solution may be prepared by any of numerous techniques known in the art or that will be developed hereafter. The high-pressure sterilization techniques are well suited to sterilize medical formulations in many different forms including a therapeutically effective compound in a dry or powder form, liquid form, gas form or dispersed as small particles or droplets in an aqueous or organic media. In an embodiment, the system to be sterilized will contain some water. The presence of water has been shown to provide particular effectiveness in obtaining reduction in active microbe load. The high-pressure sterilization techniques of the present disclosure allow for sterilization without causing a degradation of the components in the medical solution.

It is contemplated that the high-pressure sterilization techniques are suitable for use with a number of organic compounds.

The method of the present disclosure is suitable for the sterilization of medical systems and medical solutions in general. In certain embodiments, the pharmaceutically active ingredient will be such that it associates with a dispersed hydrophobic region (e.g., surfactant assembled hydrophobic phase, cyclodextrin cavity, oil droplet) in aqueous solution. Non-limiting examples of components that may be present in the medical solution include pharmaceutically active compounds, therapeutic agents, renal therapy products, diagnostic agents, cosmetics, and nutritional supplements.

The pharmaceutical active agents can be selected from a variety of known classes including, but not limited to: analgesics, anesthetics, analeptics, adrenergic agents, adrenergic blocking agents, adrenolytics, adrenocorticoids, adrenomimetics, anticholinergic agents, anticholinesterases, anticonvulsants, alkylating agents, alkaloids, allosteric inhibitors, anabolic steroids, anorexiants, antacids, antidiarrheals, antidotes, antifolics, antipyretics, antirheumatic agents, psychotherapeutic agents, neural blocking agents, anti-inflammatory agents, antihelmintics, anti-arrhythmic agents, antibiotics, anticoagulants, antidepressants, antidiabetic agents, antiepileptics, antifungals, antihistamines, antihypertensive agents, antimuscarinic agents, antimycobacterial agents, antimalarials, antiseptics, antineoplastic agents, antiprotozoal agents, immunosuppressants, immunostimulants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, hemostatics, hematological agents, hemoglobin modifiers, hormones, hypnotics, immunological agents, antihyperlipidemic and other lipid regulating agents, muscarinics, muscle relaxants, parasympathomimetics, parathyroid calcitonin, prostaglandins, radio-pharmaceuticals, sedatives, sex hormones, anti-allergic agents, stimulants, sympathomimetics, thyroid agents, vasodilators, vaccines, vitamins, and xanthines. Antineoplastic, or anticancer agents, include but are not limited to paclitaxel and derivative compounds, and other antineoplastics selected from the group consisting of alkaloids, antimetabolites, enzyme inhibitors, alkylating agents and antibiotics. The therapeutic agent can also be a biologic, which includes but is not limited to proteins, polypeptides, carbohydrates, polynucleotides, and nucleic acids. The protein can be an antibody, which can be polyclonal or monoclonal.

Diagnostic agents include x-ray imaging agents and contrast media. Examples of x-ray imaging agents include WIN 8883 (ethyl 3,5-diacetamido-2,4,6-triiodobenzoate), also known as the ethyl ester of diatrazoic acid (EEDA); WIN 67722, i.e., (6-ethoxy-6-oxohexyl-3,5-bis(acetamido)-2,4,6-triiodobenzoate; ethyl-2-(3,5-bis(acetamido)-2,4,6-triiodo-benzoyloxy)butyrate (WIN 16318); ethyl diatrizoxyacetate (WIN 12901); ethyl 2-(3,5-bis(acetamido)-2,4,6-triiodobenzoyloxy)propionate (WIN 16923); N-ethyl 2-(3,5-bis(acetamido)-2,4,6-triiodobenzoyloxy acetamide (WIN 65312); isopropyl 2-(3,5-bis(acetamido)-2,4,6-triiodobenzoyloxy)acetamide (WIN 12855); diethyl 2-(3,5-bis(acetamido)-2,4,6-triiodobenzoyloxy malonate (WIN 67721); ethyl 2-(3,5-bis(acetamido)-2,4,6-triiodobenzoyloxy)phenylacetate (WIN 67585); propanedioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo-4-(ethyl-3-ethoxy-2-butenoate) ester (WIN 68209). In an embodiment, the contrast agents may include those that are expected to disintegrate relatively rapidly under physiological conditions, thus minimizing any particle associated inflammatory response. Disintegration may result from enzymatic hydrolysis, solubilization of carboxylic acids at physiological pH, or other mechanisms. Thus, poorly soluble iodinated carboxylic acids such as iodipamide, diatrizoic acid, and metrizoic acid, along with hydrolytically labile iodinated species such as WIN 67721, WIN 12901, WIN 68165, and WIN 68209 or others may be utilized.

Other contrast media include, but are not limited to, particulate preparations of magnetic resonance imaging aids such as gadolinium chelates, or other paramagnetic contrast agents. Examples of such compounds are gadopentetate dimeglumine (Magnevist®) and gadoteridol (Prohance®).

A description of these classes of therapeutic agents and diagnostic agents and a listing of species within each class can be found in Martindale, The Extra Pharmacopoeia, Twenty-ninth Edition, The Pharmaceutical Press, London, 1989, which is incorporated herein by reference and made a part hereof. The therapeutic agents and diagnostic agents are commercially available and/or can be prepared by techniques known in the art.

Renal therapeutic agents include solutions for peritoneal dialysis (including automated or ambulatory peritoneal dialysis), hemodialysis, and/or continuous renal replacement therapy.

A cosmetic agent is any active ingredient capable of having a cosmetic activity. Examples of these active ingredients can be, inter alia, emollients, humectants, free radical-inhibiting agents, anti-inflammatories, vitamins, depigmenting agents, anti-acne agents, antiseborrhoeics, keratolytics, slimming agents, skin coloring agents and sunscreen agents, and in particular linoleic acid, retinol, retinoic acid, ascorbic acid alkyl esters, polyunsaturated fatty acids, nicotinic esters, tocopherol nicotinate, unsaponifiables of rice, soybean or shea, ceramides, hydroxy acids such as glycolic acid, selenium derivatives, antioxidants, beta-carotene, gamma-orizanol and stearyl glycerate. The cosmetics are commercially available and/or can be prepared by techniques known in the art.

Examples of nutritional supplements contemplated for use in the practice of the present disclosure include, but are not limited to, proteins, carbohydrates, water-soluble vitamins (e.g., vitamin C, B-complex vitamins, and the like), fat-soluble vitamins (e.g., vitamins A, D, E, K, and the like), and herbal extracts. The nutritional supplements are commercially available and/or can be prepared by techniques known in the art.

High-pressure sterilization equipment typically includes a sterilization chamber with temperature and pressure controls. The chamber has a lid which is closed tight while in use. The apparatus is capable of reaching pressures up to 1000 MPa or up to 1500 MPa. As used herein, ultra high pressure is pressure from about 100 MPa to about 1500 MPa and any value there between. The apparatus also has a heat source that can heat the sterilization chamber to 120° C. and above.

The method for using the apparatus includes the steps of providing a system in a desired form. In the case of medical systems, the medical system may be a powder form, solution or as an aqueous particle dispersion. In an embodiment, the medical system may include a medical preparation contained within a container which changes in volume or shape in response to pressure applied to the container. Such containers may include a flexible polymeric container or other flexible container such as a syringe barrel, cartridge for a jet injector or a metered dose inhaler. These containers will be discussed in greater detail below. The present disclosure also contemplates adding the medical preparation directly to the sterilization chamber.

The medical system may be inserted into the sterilization chamber where the medical system may be subjected to a change in pressure, a change in temperature or both simultaneously. Unlike present autoclaves for sterilizing I.V. containers and the like which only reach pressures less than 0.25 MPa, the present method subjects the preparation to pressures in excess of 0.25 MPa. In an embodiment, the medical system may be subjected to pressures from above 0.25 MPa to about 1500 MPa, or from 100 MPa to about 700 MPa and any range or combination of ranges therein.

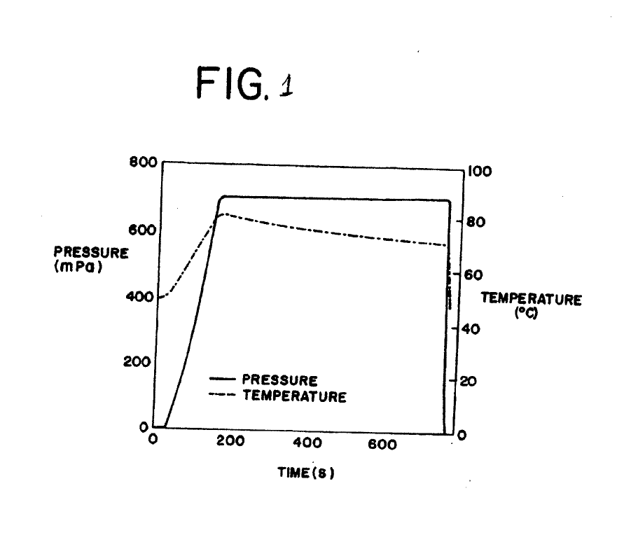

The present disclosure further includes applying temperature and pressure so as to minimize the period the medical system is exposed to temperatures in excess of 25° C. In an embodiment, the temperature of the system will be in excess of 70° C., or in excess of 90° C., or in excess of 100° C. or in excess of 120° C. and higher. Various temperature-time-pressure profiles such as the one shown in FIG. 1 can be employed to sterilize the preparation without causing a change from the stable state to the unstable state of the medical system.

In particular, FIG. 1 shows a temperature-time-pressure profile where a medical solution is exposed to pressures of about 700 MPa and energy is added to raise the temperature to about 70° C. for a period in a first cycle, followed by a second cycle of lowering the pressure to atmospheric pressure and lowering the pressure to room temperature for a period. FIG. 1 shows that the medical solution experiences rapid temperature changes during each pressure pulse. These temperature changes are induced by instantaneous adiabatic heating and cooling of the product resulting from compression and decompression, respectively. The effect of adiabatic heating during pressurization may allow a preheated solution (as described more fully hereinafter) to reach a desirable sterilization temperature, for example about 121° C. Typical times to achieve sterility are on the order of minutes, where 2 or more cycles are used.

The medical solution may be considered sterilized when the probability of a non-sterile unit is equal to or less than one in a million. This satisfies United States, European and Japanese pharmacopeia requirements.

Ultra high pressure sterilization techniques may be applied to medical solutions containing heat sensitive components to significantly reduce degradation of heat sensitive components that occurs during conventional terminal sterilization. It is known that conventional heat sterilization techniques, such as autoclaving (i.e., steam sterilization at 121° C. and about 0.58 MPa pressure, typically for up to 30 minutes), may degrade heat sensitive components such as carbohydrates, particularly when the solution is at neutral or basic pH. Glucose, a carbohydrate present in many medical solutions, becomes unstable when exposed to prolonged heat. The degradation of glucose in medical solutions results in the formation of glucose degradation products (GDPs) that may be cytotoxic, may induce pro-inflammatory activation signals, and may promote formation of advanced glycation end products (AGEs) that some studies have suggested cause vascular damage to peritoneal dialysis patients. Nonlimiting examples of GDPs include 3-deoxyglucosone (3-DG), 5-hydroxymethylfurfural (5-HMF), glyoxal, methylglyoxal (MeGly), formaldehyde, acetaldehyde, 3,4-dideoxyglucosone-3-ene (3,4-DGE), and furfural. It has been suggested that over time the damage caused by GDPs and AGEs may severely impair the filtering capability of the peritoneal membrane, which may ultimately force a PD patient to switch to a less convenient dialysis therapy such as hemodialysis. Ultra high pressure (UHP) sterilization advantageously avoids the prolonged use of high temperatures that degrade glucose and other heat sensitive components and thereby significantly reduces—and may eliminate—the formation of GDPs in sterilized medical solutions. The reduction or elimination of GDPs may retard the vascular damage to the patient's peritoneal membrane, thereby allowing the patient to continue on peritoneal dialysis. Sterilized medical solutions with a reduced amount of GDPs may also beneficially preserve residual renal function by retarding the decline of renal function.

Similarly, it is well known that many protein and peptide containing medical solutions cannot withstand conventional moist heat sterilization without significant degradation. In many cases this degradation results in diminished potency of the protein or peptide component in the solution. Accordingly, it would be advantageous to sterilize such solutions using ultra high pressure to inhibit or prevent the heat-induced degradation of the active ingredients.

In an embodiment, ultra high pressure may be used to produce a sterilized glucose-containing medical solution. The glucose-containing solution may be any solution that may be introduced into a vein or a body cavity (e.g., the peritoneal cavity) of a human or a mammal. Non-limiting examples in which patient administration of the sterilized medical solution may occur include intravenous, intra-arterial, intrathecal, intraperitoneal, intraocular, intra-articular, intradural, intraventricular, intrapericardial, intramuscular, intradermal or subcutaneous injection as is commonly known in the art. Nonlimiting examples of glucose-containing medical solutions include infusion solutions such as intravenously or subcutaneously administered solutions, and dialysis solutions. Nonlimiting examples of dialysis solutions include peritoneal dialysis solutions, continuous ambulatory peritoneal dialysis solutions, intermittent peritoneal dialysis solutions, continuous cyclic peritoneal dialysis solutions, continuous renal replacement therapy solutions, and hemodialysis solutions. In an embodiment, the glucose concentration of the infusion solution may be from about 4% to about 5% w/v. In a further embodiment, the glucose concentration may be about 4.25% w/v of the infusion solution.

In an embodiment, the infusion solution is a UP sterilized dialysis solution in which the concentration of GDPs is lower than would be present in the same solution after conventional moist heat sterilization. For example, the total GDP concentration may be less than about 25% of the GDP concentration that would be present in the same solution after conventional moist heat sterilization. In another embodiment, the sterilized solution contains less than about 20 parts per million of 3-DG. In still another embodiment, the total concentration of 5-HMF, glyoxal, methylglyoxal, 3-DG and acetaldehyde in the sterilized solution does not exceed 40 parts per million.

The method may include placing a medical system in the sterilization chamber. The medical system may include 1) a container containing 2) a medical solution. In an embodiment, the medical solution may be an infusion system. Once the medical system is placed within the sterilization chamber, the method may entail subjecting the infusion solution to ultra high pressure, i.e., a pressure from about 100 MPa to about 1500 MPa or any pressure within this range. The container may be a single chamber container containing the entire medical solution (i.e., a ready-to-use infusion solution) in the single chamber. In other words, the medical solution is not segregated into components such as a glucose solution and a buffer solution as is the case with some conventional dialysis solutions stored in multiple chamber containers. Alternatively, the container may be a multiple chamber container, each chamber holding a separate infusion solution component (e.g., an osmotic agent in one chamber and a buffer solution in another chamber.) The method may further include releasing the ultra high pressure and forming a sterile glucose solution having less that about 45 ppm total glucose degradation product. In an embodiment, the ultra high pressure applied to the medical solution system may be from about 600 MPa to about 1000 MPa, or about 650 MPa to about 800 MPa. In a further embodiment, the medical solution may be subjected to about 690 MPa pressure.

The method may include applying the ultra high pressure either continuously or intermittently to the medical solution system. In an embodiment, the ultra high pressure may be applied continuously to the container for about 1 second to about 200 seconds or any time duration within this range, for example 15, 30, 60, 90, 120 or 180 seconds. In a further embodiment, the medical solution may be subjected to an ultra high pressure from about 600 MPa to about 690 MPa for about one minute.

Alternatively, the ultra high pressure may be applied intermittently to the medical system. For example, the container may be subjected to ultra high pressure for about I second to about 60 seconds or any time duration within this range. The pressure may then be released. The ultra high pressure may then be reapplied to the container for a period from about 1 second to about 60 seconds (or any duration within the range). The amount of ultra high pressure reapplied to the container may be the same or different than the pressure level of the initial pressure application. Reapplication of UHP to the container may be repeated two, three, four, or five or more times as desired.

In an embodiment, the medical solution system may be subjected to 690 MPa pressure for about 30 seconds. The pressure may then be released. The medical solution system may then be subjected to at least one further 30-second application of about 690 MPa pressure followed by release of the pressure, for example up to about ten total pressurization cycles, thereby producing a sterilized glucose-containing solution with less than about 45 ppm total glucose degradation product. In a further embodiment, the medical solution system may be subjected to about 690 MPa for 30 seconds, the pressure may be released, and the pressure of 690 MPa may be reapplied to the system for another 30-second duration and released. The pressure of 690 MPa may be applied to the medical solution system for a third time for about 30 seconds and subsequently released. This procedure may be performed to prepare a sterilized glucose solution with a very small amount of GDPs as discussed above.

In an embodiment, the medical system may be preconditioned or otherwise pre-heated before being subjected to the ultra high pressure. For example, the medical system may be heated to a temperature from about 60° to about 100° C., or about 70° C. to about 90° C., before pressurization. This preconditioning of the container and medical solution may be accomplished by immersing the container in a heated water bath. The water bath may have a temperature from about 60° C. to about 100° C. In a further embodiment, this preconditioning procedure may be combined with the intermittent application of UHP to produce a sterilized medical solution. For example, the method may include preheating the container to a temperature from about 70° C. to about 90° C., subjecting the container to UHP, releasing the ultra high pressure, heating the container to an elevated temperature (a temperature greater than ambient temperature), and reapplying the ultra high pressure to the container to form a sterilized glucose solution having less than 45 ppm total glucose degradation product. As used herein, the term “total glucose degradation product” shall mean the sum of the concentrations of the following glucose degradation products as measured by HPLC: 3-deoxyglucosone (3-DG), 5-hydroxymethylfurfural (5-HMF), glyoxal (Gly), methylglyoxal (MeGly), and acetaldehyde.

In an embodiment, the method may include sterile filtering the medical solution prior to subjecting the solution to ultra high pressure. The filtration process may include filtering the medical solution through a filter and collecting the filtered infusion solution in a sterilized container. In an embodiment, the filter may be a 0.22 micron filter.

In an embodiment, the medical solution may be a dialysis solution having a pH from about I to about 11, for example about 4.5 to about 8.0, or about 6.0 to about 8.0, or about 6.5 to about 7.5. The dialysis solution may include the following components.

| Component | Concentration | |
|---|---|---|
| Glucose | 0-50% | |
| Glucose Polymer | 0-10% | |
| Amino Acids | 0-30% | |
| Peptides | 0-30% | |
| Calcium | 0-2 | mmol/L |
| Magnesium | 0-1 | mmol/L |
| Chloride | 0-120 | mmol/L |
| Sodium | 0-140 | mmol/L |
| Lactate | 0-40 | mmol/L |
| Bicarbonate | 0-40 | mmol/L |
| Potassium | 0-4 | mmol/L |
| Pyruvate | 0-40 | mmol/L |
| Citrate | 0-40 | mmol/L |
| Acetate | 0-40 | mmol/L |

In a further embodiment, the medical solution system may be a dialysis solution with the following composition.

| Component | Amount (g/L) |
|---|---|
| Glucose (anhydrous) | 38.6 |
| Sodium Chloride | 5.33 |
| Calcium Chloride Dihydrate | 0.254 |
| Magnesium Chloride Hexahydrate | 0.0503 |
| Sodium Lactate | 4.44 |
| pH | 5.1 |

The sterilized medical solution may include an ultra high pressure sterilized glucose solution in a container. The sterilized glucose-containing solution may include less than about 45 ppm total glucose degradation product. One of ordinary skill in the art will recognize that the ultra high pressure sterilized glucose-containing solution is a solution that has been subjected to a pressure from about 100 MPa to about 1500 MPa for a sufficient duration to achieve sterility as set forth in any of the aforementioned UHP sterilization embodiments. The total glucose degradation product may be considered another way to describe or define the amount of undegraded glucose present in the sterilized infusion solution. The total glucose degradation product is the sum of the aforementioned glucose degradation products present in the sterilized solution. Thus, it is understood that the total glucose degradation product may include none, one, some, all, or any combination of the previously listed glucose degradation products.

One of ordinary skill in the art will appreciate that there are further advantages and unique characteristics of an ultra high pressure sterilized medical system, beyond the reduced degradation of solution components as described above. For example, the short sterilization times associated with UHP sterilization—in many cases less than one minute—also result in improved solution container properties. Specifically, this short sterilization time results in a solution container that does not wrinkle, deform and/or warp to the same extent that heat sterilized containers often do. Thus, the short sterilization time not only contributes to production efficiencies, but also contributes to container longevity and improved product shelf life. UHP sterilized containers also experience less strain than conventionally sterilized containers, enabling UHP containers to maintain their original physical properties (such as mechanical modulus, optical haze, tensile strength, flexural modulus, Mooney viscosity, softening point, melting point, hardness, brittleness, etc.) for a longer period of time. Indeed, UHP sterilized containers that include peelable and/or permanent seals typically maintain substantially the same seal strengths for the respective seals pre-and post-UHP sterilization.

The container may be made from any composition suitable to withstand the forces of the ultra high sterilization process. In an embodiment, the container may be made of any flexible, PVC or non-PVC polymeric composition as described in detail below. Alternatively, the container may be constructed of semi-rigid materials but incorporate structural features responsive to the application of ultra high pressure, as described further below. The sidewalls of the container may be a single layer or may be a multiple layer structure. In an embodiment, the container may have an interior storage volume from about 100 milliliters to about 5 liters or any volume therebetween.

In an embodiment, the medical solution may be a UHP sterilized dialysis solution. The dialysis solution may include an osmotic agent selected from the group consisting of glucose, glucose polymer, and combinations thereof. The osmotic agent may be present in an amount from about 1 % to about 50% by weight of the solution. The solution may have a pH from about 6.0 to about 8.0, or from about 6.7 to about 7.5. The solution may contain less than about 45 ppm total glucose degradation product.

The dialysis solution may be ready-to-use; i.e., requiring no mixing of segregated components, no addition of other components, or no further sterilization, prior to administration to a patient. In a further embodiment, the dialysis solution may include additional components in addition to the osmotic agent such as amino acids, buffers, or electrolytes. Nonlimiting examples of other dialysis components may include 0-30% by weight of at least one amino acid, peptide, or protein; 0-2 mmol/L calcium, 0-1 mmol/L magnesium, 0-120 mmot/L chloride, 0-140 mmol/L sodium, 0-40 mmol/L lactate, 0-40 mmol/L bicarbonate, 0-4 mmot/L potassium, 0-40 mmol/L citrate, 0-40 mmol/L acetate, and any combination thereof.

In an embodiment, the sterilized dialysis solution may be substantially or entirely free of any precipitate. The sterilized dialysis solution may also be clear—i.e., have no, or substantially no, cloudiness and/or turbidity.

In an embodiment, the medical system may include a single chamber container, such as the containers set forth in FIG. 2 . In other words, the UHP sterilized medical solution may be a ready-to-use medical solution disposed in a single chamber container. The container may also be UHP sterilized simultaneously with the medical solution. In an embodiment, the entire glucose-containing medical solution is disposed in the single chamber of the container.

Certain medical solution systems, and dialysis solutions in particular, employ a multiple chamber container for separately containing two infusion solution components, e.g. a buffer component and a glucose component, each solution component being contained in a separate chamber of the multiple chamber container. For example, a peritoneal dialysis solution may be provided as a glucose/electrolyte component at an acidic pH and a basic buffer solution, such that the combined solutions have a physiologically acceptable pH. It is known that providing the glucose at a high concentration and in an acidic environment limits the degradation of the glucose during conventional sterilization. Not wishing to be bound to any particular theory, segregation of a dialysis solution into separate container chambers prevents reaction or precipitation of solution ingredients that would otherwise occur during conventional heat sterilization processes in the event the dialysis solution remained intact or otherwise unseparated.

It is often desirable to provide such two-part dialysis solutions formulated so that, when combined for administration, the mixed solution will have a pH close to physiologic pH, i.e. between about 6.0 and about 8.0. On the other hand, certain advantages arise from providing a two-part dialysis solution in which the glucose component is maintained at low pH (for example from about 1.9 to about 4, or from about 3 to about 3.5) during sterilization, even if the pH of the mixed solution is below about 6.0. In particular, such solutions should exhibit significantly reduced formation of glucose degradation products during sterilization relative to comparable formulations sterilized in single-chamber containers. Table 1 below describes an exemplary two-part dialysis solution system in which the mixed solution is very similar to a commercial one-part solution (DIANEAL 4 from Baxter Healthcare Corporation). Although the exemplary system contains about 4.25% dextrose, it is of course possible to formulate similar systems at reduced dextrose concentrations, for example 2.5% or 1.25% dextrose. Persons skilled in the art will appreciate that a number of further variations on the exemplary system are also possible. These may include, without limitation, replacing some of the dextrose with another osmotic agent; replacing all or part of the lactate buffer with one or more alternative, physiologically acceptable buffers such as bicarbonate, acetate, citrate or pyruvate; altering the distribution of the sodium, calcium and/or magnesium electrolytes between the two solutions, and the like.

TABLE 1
Formulation of a Two-Part Dialysis Solution (4.25% dextrose,
calcium and magnesium in dextrose concentrate).

| | Dextrose | Buffer | Mixed |
|---|---|---|---|
| Component (g/L) | Concentrate | Concentrate | Solution |
| Dextrose, anhydrous | 106.5 | 0 | 38.6 |
| CaCl₂•2 H₂O | 0.71 | 0 | 0.26 |
| MgCl₂•6 H₂O | 0.14 | 0 | 0.05 |
| NaCl | 7.71 | 4.05 | 5.38 |
| NaLactate | n/a | 7.04 | 4.48 |
| pH adjuster | HCl | NaOH | N/A |
| pH | 3.1-3.5 | 7.4-7.8 | 5.8-6.0 |
| Solution volume | 725 mL | 1275 mL | 2000 mL |

There are, however, numerous advantages of a ready-to-use UHP sterilized medical solution disposed in a single-chamber container. For example, multiple-chamber containers can present challenges in terms of keeping the component solutions separate but ensuring proper mixing before infusion. Consequently, it is highly desirable to provide the dialysis solution in a single-chamber, ready-to-use container. The present ultra high pressure sterilized medical solution obviates the need to segregate a dialysis solution into individual components as no precipitates are formed. Accordingly, provision of the UHP sterilized dialysis solution avoids the need to separate buffer and glucose components of the dialysis solution in separate container chambers because the ultra high pressure sterilized dialysis solution exhibits no post-sterilization precipitates and/or cloudiness.

A further advantage of the UHP sterilized medical solution is that it may have a higher pH than conventional dialysis solutions. Not wishing to be bound to any particular theory, conventional dialysis solutions typically have a pH in the range of about 5.0-5.5 in order to stabilize the glucose component and prevent the formation of toxic Maillard reaction products during heat sterilization. By avoiding conventional heat sterilization, the present UHP sterilized dialysis solution provides a more stable system. Accordingly, in an embodiment, the pH of the sterilized dialysis solution may be from about 6.0 to about 8.0, or about 6.5 to about 7.5, or any pH value therebetween. Dialysis solutions having a pH in this “physiologic” range are advantageous because patients experience less pain upon infusion of such solutions compared with typical dialysis solutions having a pH from about 5.0 to about 5.5.

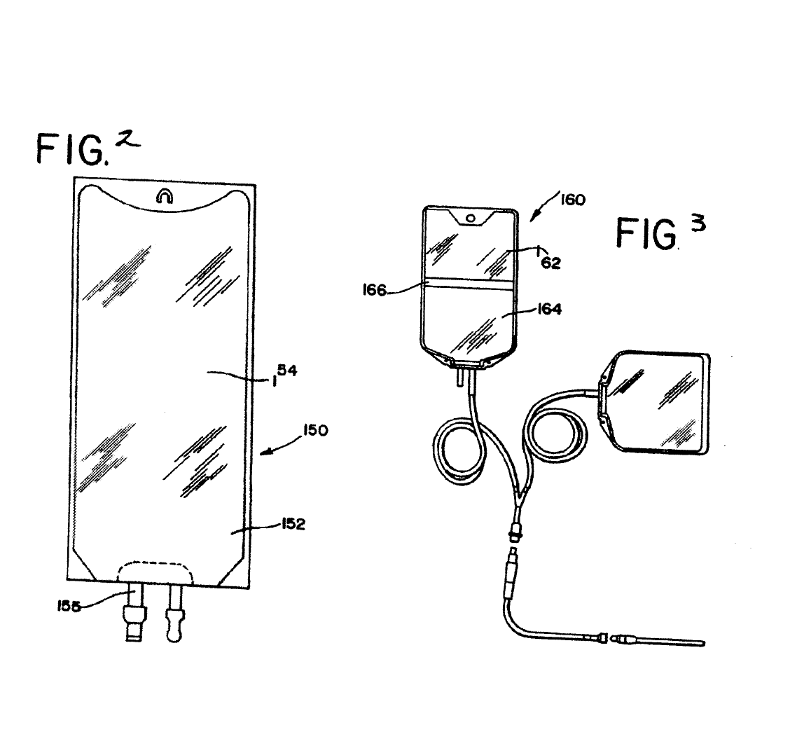

It is understood, however, that conventional dialysis solution systems utilizing multiple chamber containers (and corresponding separated/isolated solution components) may also be sterilized using the aforementioned ultra high pressure sterilization techniques. In an embodiment, the medical system may include a multiple chamber container, such as container 160 in FIG. 3. In this embodiment, a dialysis solution may be segregated into an osmotic agent disposed in compartment 162 and a buffer component contained in compartment 164, compartments 162, 164 separated by peel seal 166.

In an embodiment, the osmotic agent component may have a pH of about 1.5 to about 5.5, for example from about 2.0 to about 4.5; and the buffer component may have a pH from about 6.0 to about 10.0, for example from about 6.0 to about 8.0.

In an embodiment, the sterilized glucose-containing solution may contain less than 2.34 ppm acetaldehyde, or from about 0 ppm to about 2.34 ppm (or any amount within this range) acetaldehyde, or from about 0.2 ppm to about 0.45 ppm acetaldehyde. In an embodiment, the UHP sterilized glucose-containing solution may contain less than 0.29 ppm formaldehyde, or from about 0 ppm to about 0.29 ppm formaldehyde (or any amount within this range), or from about 0.05 ppm to about 0.10 ppm formaldehyde.

In an embodiment, the UHP sterilized glucose-containing solution may contain less than about 14 ppm 3-deoxyglucosone, or about 0 ppm to about 14 ppm 3-deoxyglucosone (or any amount within this range), or less than 7 ppm 3-deoxyglucosone. In a further embodiment, the UHP sterilized infusion solution may include less than 1 mg 3-deoxyglucosone per gram of glucose, or less than 0.5 mg 3-deoxyglucosone per gram of glucose, or less than 0.2 mg 3-deoxyglucosone per gram of glucose.

In an embodiment, the UHP sterilized glucose-containing solution may contain less than about 2.3 ppm methylglyoxal, or about 0 ppm to about 2.3 ppm methylglyoxal (or any amount within this range), or less than about 0.5 ppm methylglyoxal, or less than about 0.3 ppm methylglyoxal. In an embodiment the UHP sterilized glucose-containing solution may contain substantially no or no methylglyoxal.

In an embodiment, the UHP sterilized glucose-containing solution may contain less than about 2.3 ppm glyoxal, for example about 0 ppm to about 2.3 ppm glyoxal (or any amount within this range), or less than about 0.5 ppm glyoxal, or less than about 0.3 ppm glyoxal.

In an embodiment, the UHFP sterilized glucose-containing solution may contain no, or substantially no, 3,4-dideoxyglucosone-3-ene and/or furfural. It is understood that the discussion of GDPs applies to any glucose containing medical solution (infusion solutions, dialysis solutions) presented herein.

An example of a UBP sterilized medical solution is set forth below.

A 1% itraconazole nanosuspension exposed to a high-pressure sterilization cycle is currently being tested for sterility. In saline, the effect of high-pressure sterilization on the lethality of Bacillus stearothernophilus has already been demonstrated (using the most heat-resistant of the strains mentioned to have demonstrated high moist heat resistance with respect to bioburden—see reference ANSI/AAMI/ISO 11134-1993, Sterilization of health care products—Requirements for validation and routine control—Industrial moist heat sterilization. American National Standard developed by the Association for the Advancement of Medical Instrumentation and approved by the American National Standards Institute, page 12, section A.6.6.). Test and control units inoculated with at least one million spores of Bacillus stearothermophilus were subjected to two different processes—the first process used a pressure of approximately 600 MPa for 1 minute and the second used a pressure of approximately 600 MPa for six 10-second cycles. The initial and highest temperatures in both processes were 90° C. and 121° C., respectively. No survivors were found in the saline solutions for both processes (see Table 2). It is anticipated that similar results will be found when the 1% itraconazole nanosuspension is inoculated and sterilized.

TABLE 2
Lethality of Bacillus stearothermophilus in
Two High-Pressure Sterilization Processes

| Solution | Sterilization Conditions | CFU/ml |
|---|---|---|
| Saline Solution 1 - | None | 1.9 × 10⁶ |
| Control | | |
| Saline Solution 1 - | 600 MPa, One 1-minute cycle, | 0 |
| Sterilized | Initial Temperature = 90° C., High | |
| | Pressure Temperature = 121° C. | |
| Saline Solution 2 - | None | 3.7 × 10⁶ |
| Control | | |
| Saline Solution 2 - | 600 MPa, Six 10-second cycles, | 0 |
| Sterilized | Initial Temperature = 90° C., High | |
| | Pressure Temperature = 121° C. | |

Tables 3, 4 and 5 below compare the amount of GDPs present in conventional heat sterilized infusion solutions with the GDPs present in UHP sterilized infusion solutions. Specifically, samples of a commercial peritoneal dialysis solution (DIANEAL PD-2 Solution with 4.25% Dextrose from Baxter Healthcare Corporation) in flexible PVC containers were sterilized using conventional moist heat sterilization and under several different ultra high pressure sterilization conditions. A significant reduction in GDPs was observed in the UHP sterilized samples relative to the conventionally sterilized control.

TABLE 3
Comparison of GDP Levels in 4.25% glucose solution:
Conventional Heat Sterilized vs. UHP Sterilized

| | | 5-HMF | Acetaldehyde | Formaldehyde | 3-DG | Glyoxal | MeGly |
|---|---|---|---|---|---|---|---|
| Sample | pH | (ppm) | (ppm) | (ppm) | (ppm) | (ppm) | (ppm) |
| Conventional Heat | 5.1 | 4.3 | 2.34 | 0.29 | 42.6 | 2.35 | 0.8 |
| Sterilized | | | | | | | |
| UHP Sterilized | 5.1 | 0.3 | 0.30 | 0.09 | 8.53 | 0.1 | ND |
| 690 MPa | | | | | | | |
| (2 × 30 sec) | | | | | | | |
| UHP Sterilized | 5.1 | 0.5 | 0.23 | 0.09 | 7.85 | 0.1 | ND |
| 690 MPa 60 sec | | | | | | | |
| UHP Sterilized | 5.1 | 0.3 | 0.43 | 0.09 | 13.7 | 0.1 | ND |
| 690 MPa | | | | | | | |
| (3 × 30 sec) | | | | | | | |

ND = not detected

TABLE 4
Additional Testing Data

| Sample | 3-DG (ppm) | Glyoxal (ppm) | MeGly (ppm) |
|---|---|---|---|
| Conventional Heat | 42.8 | 1.0 | 0.4 |
| Sterilized | | | |
| UHP Sterilized | 4.4 | 0.1 | ND |
| 600 MPa for 1 | | | |
| minute) | | | |
| UHP Sterilized | 3.0 | 0.1 | ND |
| 690 MPa for 5 | | | |
| seconds | | | |

TABLE 5
Additional Testing Data

| Sample | Sterilization Method | 3-DG (ppm) |
|---|---|---|
| DIANEAL (4.25% glucose) | Conventional Heat | 42.7 |
| | Sterilized | |
| DIANEAL in single bag | UHP Nominal | 4.4 |
| | (600 MPa for 60 seconds) | |
| DIANEAL in single bag | UHP Severe | 7.8 |
| | 690 MPa for 60 seconds | |
| DIANEAL adjusted to pH 6.5 | UHP Nominal | 8.2 |
| 2.1% glucose with 0.28% | UHP Nominal | 4.0 |
| amino acids | | |

The data in Table 5 reflect approximately a tenfold reduction in the concentration of 3-deoxyglucosone relative to conventional moist heat sterilization.

Various containers, such as those used as medical devices (e.g., for pharmaceutical administration, renal dialysis and blood collecting/processing can be sterilized by methods of the present disclosure. Examples of such containers include, but are not limited to, fluid administration sets (including those containing syringes), blood collection assemblies (e.g., blood pack units), disposable assemblies for automated blood processing, dialyzer assemblies, and peritoneal dialysis bags, catheters and assemblies. Typically such systems will contain a fluid transfer member (e.g., tubing).

FIG. 2 shows a flowable materials container 150 having two sidewalls 152 defining a chamber 154 therebetween. An access member 155 provides for sterile access to the contents of the container. FIG. 3 shows a multiple chamber container 160 having first and second chambers 162, 164 connected by a peel seal 166. Such multichamber containers are particularly suitable for storing a liquid in one chamber and a powder in the second chamber or liquid in both chambers. The peel seal allows for mixing of the components just before use. Suitable multiple chamber containers include, but are not limited to, those disclosed in U.S. Pat. Nos. 5,577,369 and 6,017,598, incorporated herein by reference and made a part hereof. For example, the container may be a sealed fluid container, a syringe and/or a sealed tubing.

In an embodiment, the sidewalls are made from a non-PVC containing polymer. The sidewalls can be formed from a monolayer or multilayer structure. In an embodiment, the sidewalls are non-oriented and are not considered heat-shrinkable films.

Suitable non-PVC containing polymers for forming the sidewalls of the container are disclosed in commonly assigned U.S. Pat. Nos. 5,998,019; 6,461,696; 6,964,798; 6,969,483; European Patent No. EP 1 139 969; and International Patent Publication No. WO 2005/040268 A1, each of which is incorporated herein by reference and made a part hereof. It will be apparent to those of ordinary skill in the art that other heat-resistant non-PVC container films known in the art may also be used to form the sidewalls of the container. For example, additional suitable films are disclosed in U.S. Pat. Nos. 6,027,776; 5,695,840; and 4,643,926.

The high-pressure sterilization techniques of the present disclosure are also suitable for sterilizing empty drain bags for renal CAPD applications such as the container disclosed in U.S. Pat. No. 6,004,636, which is incorporated herein by reference and made a part hereof. Other containers suitable for terminal sterilization using the high-pressure sterilization techniques of this disclosure include flexible cell culture containers such as those disclosed in U.S. Pat. Nos. 5,935,847; 4,417,753; and 4,210,686 which are incorporated in their entirety herein by reference and made a part hereof. Protein compatible films and containers such as those disclosed in U.S. Pat. No. 6,309,723, which is incorporated herein by reference and made a part hereof, can also be sterilized using the high-pressure sterilization techniques disclosed herein. Further, the sterilization techniques are also suitable for sterilizing containers for containing oxygen sensitive compounds such as deoxygenated hemoglobin as is disclosed in U.S. Pat. No. 6,271,351, which is incorporated herein by reference and made a part hereof. Because the sterilization techniques require exposing such containers to temperatures greater than 100° C. only for a short time, many containers that are unsuitable for terminal sterilization using standard techniques of exposing the container to steam at 121° C. for 1 hour can be terminally sterilized with the high pressure techniques of the present disclosure.

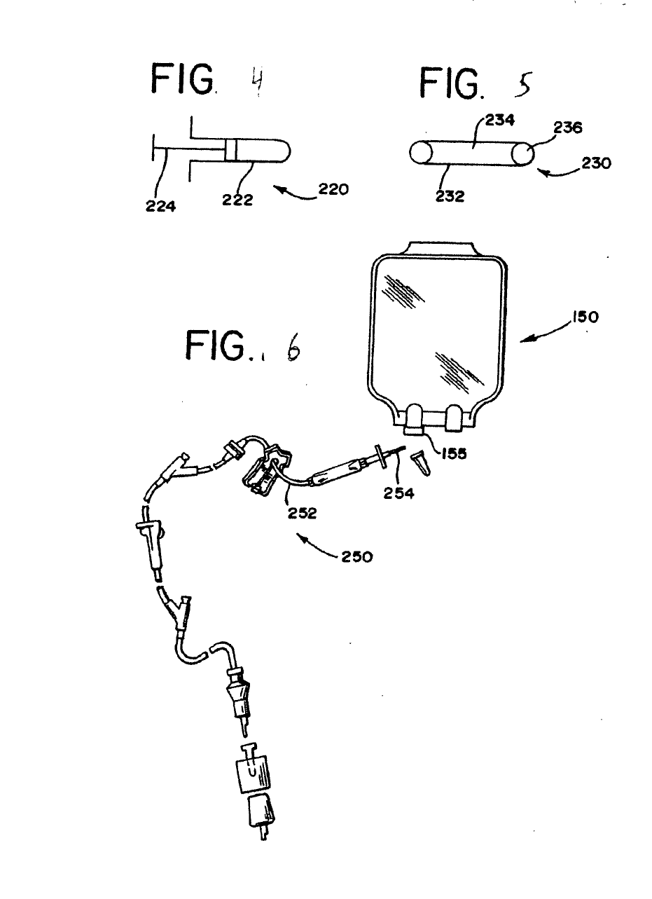

FIG. 4 shows a syringe 220 having a barrel 222 and a plunger 224 as is well known in the art. The syringe 220 can be fabricated from the materials described above. The syringe barrel can be filled with one of the dispersions or dry powder of the pharmaceutical compound and then autoclaved as described above. The syringe barrel and preferably both the barrel and the plunger must be capable of changing volume in response to an increased pressure and both parts 222 and 224 must have sufficient heat distortion resistance to be capable of withstanding the terminal sterilization process of this disclosure.

FIG. 5 shows a cartridge 230 or insert having a body 232 defining a chamber 234. The chamber 234 is sealed with an end cap 236 or a pair of end caps if necessary. The cartridge can be inserted into a delivery device such as a jet injector such as those set forth in U.S. Pat. No. 6,132,395, or in other delivery device that is capable of accessing the contents of the chamber 234 and delivering the contents for use.

FIG. 6 shows a fluid access device 250 having a medical tubing 252 and an access device 254. The access device can be an object for piercing an access member 155 (FIG. 2) or can be adapted to dock or otherwise connect to the syringe barrel 222 (FIG. 4) to convey fluid from the container used for sterilization to delivery to a patient or to another device used to deliver the composition to a patient.

The present disclosure provided sterilized medical systems such as medical solutions, infusion solutions, pharmaceutical preparations, container, containers containing sterile medical solutions, the systems having been sterilized by optionally supplying heat to the product and pressurizing the product to a pressures of greater than 0.25 MPa. The present disclosure also provides sterile medical solutions such as glucose-containing solutions with low or very low amounts of glucose degradation product.

It should be understood that various changes and modifications to the presently preferred embodiments described herein will be apparent to those skilled in the art. Such changes and modifications can be made without departing from the spirit and scope of the present disclosure and without diminishing its intended advantages. It is therefore intended that such changes and modifications be covered by the appended claims.

1. A method of producing a sterile dialysis solution comprising the steps of: providing an aqueous dialysis solution comprising an osmotic agent and a buffer in a flexible container; pressurizing the solution and container to a hydrostatic pressure of about 100 MPa to about 1500 MPa; and releasing the hydrostatic pressure, thereby producing a sterile solution within the container.

2. The method of claim 1, wherein substantially all of the dialysis solution is disposed within one compartment of the container during the pressurization step.

3. The method of claim 1, wherein the container comprises separate compartments containing the osmotic agent and the buffer, the container being adapted to permit selective fluid communication between the compartments.

4. The method of claim 1, wherein the dialysis solution is substantially free of oxidation inhibiting agents.

5. The method of claim 1, wherein the osmotic agent is selected from glucose, polymers thereof and mixtures thereof, and whereby the sterile dialysis solution comprises a lower concentration of glucose degradation products than would be present in the aqueous dialysis solution following terminal moist heat sterilization.

6. The method of claim 5, wherein the concentration of glucose degradation products in the sterile dialysis solution is less than about 25 percent of the concentration of glucose degradation products that would be present in the aqueous dialysis solution following terminal moist heat sterilization.

7. The method of claim 1, wherein the osmotic agent comprises glucose and the sterile solution comprises less than about 20 parts per million of 3-deoxyglucosone.

8. The method of claim 1, wherein the osmotic agent is selected from glucose, polymers thereof, and mixtures thereof, and wherein the total concentration of 5-hydroxymethylfurfural, glyoxal, methylglyoxal, 3-deoxyglucosone, and acetaldehyde in the sterile dialysis solution does not exceed 40 parts per million.

9. The method of claim 1, further comprising heating the dialysis solution to a temperature from about 70° C. to about 99° C. before pressurizing the container.

10. The method of claim 1 wherein the pressure is from about 600 MPa to about 1000 MPa.

11. The method of claim 1 further comprising performing said pressurizing and releasing steps at least twice.

12. The method of claim 1 further comprising further heating the solution.

13. The method of claim 1 wherein the step of pressurizing has a duration from about I second to about 200 seconds.

14. A method of preparing a dialysis solution comprising:
providing first and second component solutions in discrete chambers of a multiple chamber container adapted to permit selective fluid communication between said chambers, said first component solution having a pH of about 1.5 to about 5.5 and comprising up to about 50 weight percent glucose, glucose polymer, or a mixture thereof, and said second component solution comprising a buffer concentrate having a pH of about 6.0 to about 10.0, wherein said first and second component solutions when mixed form a ready-to-use dialysis solution having a pH of about 4.5 to about 8.0; and
subjecting said multiple chamber container to a hydrostatic pressure from about 100 MPa to about 1500 MPa for 1 to about 300 seconds.

15. The process of claim 14, wherein the first component solution has a pH of about 1.9 to about 4.5.

16. The process of claim 14, wherein the second component solution has a pH of about 6 to about 8.

17. The process of claim 14, wherein the second component solution has a pH of about 8.5 to about 10.0.

18. The process of claim 14, further comprising the step of preheating the container to a temperature of about 70° C. to about 99° C. before subjecting the container to the hydrostatic pressure.

19. A method of preparing a dialysis solution comprising:
providing first, second and third component solutions in discrete chambers of a multiple chamber container, said container adapted to permit selective fluid communication between said chambers,
said first component solution having a pH of about 3.0 to about 6.0 and comprising up to about 50 weight percent glucose, glucose polymer, or a mixture thereof,
said second component solution comprising a buffer concentrate having a pH of about 6.5 to about 10.0, and
said third component solution having a pH of about 1.5 to about 4.5,
wherein upon mixing said first, second and third component solutions a ready-to-use dialysis solution having a pH of about 4.5 to about 8.0 is formed; and
subjecting said multiple chamber container to a hydrostatic pressure from about 100 MPa to about 1500 MPa for 1 to about 300 seconds.

20. The process of claim 19, wherein the first component comprises a glucose polymer.

21. The process of claim 19, wherein the third component solution comprises glucose.

22. The process of claim 19, further comprising the step of preheating the container to a temperature of about 70° C. to about 99° C. before subjecting the container to the hydrostatic pressure.

23. A method of preparing a dialysis solution comprising:
providing first and second component solutions in discrete chambers of a multiple chamber container, said container adapted to permit selective fluid communication between said chambers,
said first component solution comprising up to about 50 weight percent glucose, glucose polymer, or a mixture thereof, and said second component solution comprising a buffer concentrate,
wherein upon mixing said first and second component solutions a ready-to-use dialysis solution having a pH of about 4.5 to about 8.0 is formed; and
subjecting said multiple chamber container to a hydrostatic pressure from about 100 MPa to about 1500 MPa for 1 to about 300 seconds.

24. The process of claim 23, further comprising the step of preheating the container to a temperature of about 70° C. to about 99° C. before subjecting the container to the hydrostatic pressure.

25. A terminally sterilized dialysis solution packaged in a single-chamber primary container during sterilization, the solution comprising:
an osmotic agent comprising glucose, the osmotic agent present in an amount from about 1% to about 50% by weight of the solution, the sterilized solution having a pH between about 5.0 and about 6.0 and containing less than about 5 ppm of 3-deoxyglucosone.

26. A terminally sterilized dialysis solution packaged in a single-chamber primary container during sterilization, the solution comprising:
an osmotic agent comprising glucose, the osmotic agent present in an amount from about 1% to about 50% by weight of the solution, the sterilized solution having a pH between about 6.0 and about 8.0 and containing less than about 40 ppm of 3-deoxyglucosone.

27. The dialysis solution of claim 26 wherein the pH is between about 6.5 and about 7.5.

28. The dialysis solution of claim 26 wherein the concentration of glucose in the solution is approximately 1.5% to about 5% w/v.

29. The dialysis solution of claim 26 wherein the solution further comprises at least one buffer selected from the group consisting of lactate, citrate, acetate, pyruvate, bicarbonate, and mixtures thereof.

30. The dialysis solution of claim 29 wherein the buffer comprises bicarbonate.

31. The dialysis solution of claim 26 further comprising 0-30% by weight of at least one amino acid, peptide, or protein, 0-2 mmol/L calcium, 0-1 mmol/L magnesium, 0-4 mmol/L potassium, 0-120 mmol/L chloride, 0-140 mmol/L sodium, and 0.1-40 mmol/L of one or any combination of lactate, bicarbonate, citrate, pyruvate and acetate.

32. The dialysis solution of claim 26 wherein the solution includes less than about 14 ppm 3-deoxyglucosone.

33. The dialysis solution of claim 26 wherein the solution contains less than about 7 parts per million of 3-deoxyglucosone.

34. The dialysis solution of claim 33 wherein the solution contains substantially no 3-deoxyglucosone.

35. The dialysis solution of claim 26 wherein the solution includes less than about 2.3 ppm glyoxal.

36. The dialysis solution of claim 26 wherein the solution includes less than about 1 ppm methylglyoxal.

37. The dialysis solution of claim 26 wherein the solution includes less than about 2.3 ppm acetaldehyde.

38. The dialysis solution of claim 26 wherein the solution contains substantially no furfural.

39. A method of treating renal disease comprising the step of providing a sterile dialysis solution in a flexible container, said solution comprising at least one osmotic agent, at least one buffer and at least one electrolyte, the solution and container having been pressurized together at a pressure of about 100 MPa to about 1500 MPa.

40. The method of claim 39, further comprising the step of infusing the sterile solution into a patient in need thereof.

41. The method of claim 39, wherein the flexible container contains a mixture of the buffer and osmotic agent during pressurization.

42. The method of claim 41, wherein the container comprises only one solution compartment.

43. The method of claim 39, wherein the container comprises separate compartments for the osmotic agent and the buffer.

44. The method of claim 39, wherein the solution is substantially free of oxidation inhibiting agents.

45. A terminally sterilized medical solution in a single-chamber container, the sterilized solution consisting essentially of water, glucose or a polymer thereof, 0.1-30% by weight of at least one of an amino acid, a peptide, or a protein; 0-2 mmolIL calcium, 0-1 mmoUL magnesium, 0-120 mmol/L chloride, 0-140 mmol/L sodium, 0-4 mmol/L potassium, and 0-40 mmot/L of at least one lactate, bicarbonate, citrate, acetate, pyruvate, or mixtures thereof, the solution having a pH from about 6.0 to about 8.0 and containing less than about 40 ppm total glucose degradation product.

46. The medical solution of claim 45 wherein the pH is from about 6.5 to about 7.5.

47. The medical solution of claim 45 wherein the solution contains from about 1% to about 50% by weight of an osmotic agent selected from the group consisting of glucose, glucose polymers, and combinations thereof.

48. The medical solution of claim 45 wherein the solution includes less than about 40 ppm 3-deoxyglucosone.

49. The medical solution of claim 45 wherein the solution includes less than about 2.3 ppm glyoxal.

50. The medical solution of claim 45 wherein the solution includes less than about 0.75 ppm methylglyoxal.

51. The medical solution of claim 45 wherein the solution includes less than about 2.3 ppm acetaldehyde.

52. A terminally sterilized dialysis solution in a single-chamber container, said solution consisting essentially of glucose, water, 0-10% by weight an amino acid, 0-30% by weight a peptide, 0-2 mmol/L calcium, 0-1 mmol/L magnesium, 0-120 mmol/L chloride, 0-140 mmol/L sodium, 0-4 mmol/L potassium, and 0-40 mmol/L of one or a combination of lactate, bicarbonate, citrate, and acetate, the solution having a pH from about 6.0 to about 8.0 and containing less than about 45 ppm total glucose degradation product.

53. A method for sterilizing a medical system comprising:
subjecting a container containing a glucose solution to a pressure from about 100 MPa to about 1500 MPa; and
releasing the pressure,
thereby forming a sterile glucose solution containing less than about 40 ppm total glucose degradation product.

54. The method of claim 53, whereby the sterile glucose solution contains less than about 20 ppm of 3-deoxyglucosone.

55. The method of claim 53 further comprising heating the glucose solution to a temperature from about 70° C. to about 99° C. before subjecting the container to the pressure.

56. The method of claim 53 wherein the pressure is from about 600 MPa to about 1000 MPa.

57. The method of claim 53 further comprising performing said subjecting and releasing steps at least twice.

58. The method of claim 53 further comprising further heating the glucose solution.

59. The method of claim 53 wherein the step of subjecting has a duration from about 1 second to about 200 seconds.

60. The method of claim 53 wherein the glucose solution is an infusion solution and includes at least one osmotic agent selected from the group consisting of glucose, glucose polymer, amino acids, peptides, and combinations thereof; and at least one further component selected from the group consisting of calcium, magnesium, chloride, sodium, lactate, bicarbonate, potassium, citrate, acetate, and combinations thereof.

61. The method of claim 53 wherein the container is a single chamber container.

62. A two-part dialysis solution product comprising:
first and second component solutions in discrete chambers of a multiple chamber container, said container adapted to permit selective fluid communication between said chambers,
said first component solution having a pH of about 1.9 to about 4.0 and comprising up to about 50 weight percent glucose, glucose polymer, or a mixture thereof, and
said second component solution comprising a buffer concentrate,
wherein upon mixing said first and second component solutions a ready-to-use dialysis solution having a pH of about 4.5 to about 6.0 is formed.

63. The dialysis solution according to claim 62, wherein the first component solution has a pH of about 3.1 to about 3.5.

64. The dialysis solution according to claim 62, wherein the buffer concentrate comprises a buffer selected from lactate, citrate, acetate, pyruvate, bicarbonate and mixtures thereof.

65. The dialysis solution according to claim 62, wherein the ready-to-use dialysis solution has a pH of about 5.8 to about 6.0.

66. The dialysis solution according to claim 62, wherein the ready-to-use dialysis solution contains less than about 40 parts per million by weight of total glucose degradation product.
